(19) United States
(12) Patent Application Publication
(10) Pub. No.: US 2017/0209709 A1
(43) Pub. Date: Jul. 27, 2017

(54) LIGHT TREATMENT APPARATUS

(71) Applicant: Shenzhen Peninsula Medical Co., Ltd., Shenzhen (CN)

(72) Inventor: XIAOBING LEI, Shenzhen (CN)

(73) Assignee: Shenzhen Peninsula Medical Co., Ltd., Shenzhen (CN)

(21) Appl. No.: 15/480,773

(22) Filed: Apr. 6, 2017

(30) Foreign Application Priority Data
Sep. 9, 2016 (CN) 201610812336.7

(51) Int. Cl. A61N 5/06

(52) U.S. Cl. CPC A61N 5/0616; A61N 2005/067

(57) ABSTRACT

An apparatus for pigment and vascular treatment, including: a treatment main portion including a low-peak-power laser configured to output a laser light spot; a controller configured to perform laser treatment control; and a treatment handle connected to the treatment main portion via an optical path and an electrical circuit; the treatment handle including: a scanning resonance optical structure optically coupled to the laser via the optical path and configured to cause the laser to have a scanning output in accordance with a program of pulse widths and light spot positions set by the controller; and a treatment window member coupled to an output end of the scanning resonance optical structure and configured to adjust a surface area of the laser light spot on a treatment surface; wherein the treatment window member is disposed between a field lens of the scanning resonance optical structure and the treatment surface.

### CROSS-REFERENCE TO RELATED APPLICATION

The present application claims priority to Chinese Patent Application No. CN 201610812336.7 filed on Sep. 9, 2016, the disclosures of which is hereby incorporated by reference in its entirety.

### BACKGROUND

Pigments/blood vessels can be treated with light of a particular wavelength, and the therapeutic mechanism is based on the selective absorption by the target of light at particular wavelength bands. In practical applications, laser can be used as a light source for treatment, and the laser pulse width is usually in the range of ns and ms. Lasers that can be narrowed to such pulse widths are usually only solid-state lasers or dye lasers. In addition to the need for shorter pulse widths, pigment/vascular therapy may also require a higher energy density, usually of 30-100 J/cm². As is well known, the higher the power of the laser, the higher the cost. Even a relatively small difference between lasers can sometimes dictate that the price differences can be several times. Therefore, the existing pigment/vascular treatment lasers have high cost, and there is also generally a high failure rate during the use of the lasers, greatly limiting the applications of these lasers.

The interactions between light and tissues have been well studied. For example, based on the biological effects of different ultraviolet (UV) light, UV is divided into four wavelength bands. UVB band has wavelengths 275˜320 nm, also known as having the red erythema effect of ultraviolet light. Having a medium penetration capability, its shorter wavelengths will be absorbed by the transparent glass, and most of the ultraviolet light contained in the sun light is absorption by the ozone layer, leaving only less than 2% to reach the Earth's surface, and particularly strong in the summer and afternoon. UVB has the erythema effect on the human body, which can promote the body's mineral metabolism and the formation of vitamin D. However, long-term or excessive exposure can tan the skin, and cause swelling and peeling. UV light lamps and plant growth lamps employ special UV-transmission glass (but opaque for light with wavelengths lower than 254 nm), and fluorescent material peaking in the vicinity of 300 nm.

In modern medicine, UVB has been found to have some good medical therapeutic effects, such that UVB has been employed to treat some skin diseases. Accordingly, more and more medical devices are developed employing UVB.

In UVB treatment, generally the 308-nm excimer light is used, because of its high optical power, and better wavelength monochromaticity. Such UV light therapy has achieved good clinical applications. Relatively speaking, its shortcomings includes the small spot output, the slow treatment speed that waste the doctors' and patients' time. Meanwhile, in the current market, UVB large-area treatment generally employs 311-nm UV lamps, with low optical power output, and power wavelength monochromatic.

### SUMMARY

In view of the above-mentioned drawbacks of the conventional devices, it is an object of the present disclosure to provide an apparatus for realizing pigment and vascular treatment using a low-peak-power laser for solving the problems that expensive solid-state lasers are required in conventional pigment treatment, resulting in high cost and high failure rate.

It is another object of the present disclosure to provide a method for solving the problem that the spot light output is small and that the treatment speed is slow in the conventional UVB treatment devices. The embodiments disclosed herein can also solve the problems in the current technologies of the UVB large-area treatment, including that the optical power output is very low, and the light wavelength monochromatic problems.

In order to achieve the above objects and other related objects, the present disclosure provides the following technical solutions.

An apparatus for achieving pigment and vascular therapy using a low-peak-power laser, including: a treatment main portion including a laser for outputting a laser, and a control system for performing laser treatment control, wherein the laser is a semiconductor laser; a treatment handle connected to the treatment main portion via one or more optical paths and electrical circuits. The apparatus can further include a treatment window member being connected to an output end of a scanning resonance mirror mechanism. In some embodiments, the treatment window member is configured to adjust a surface area of the laser light spot on the treatment surface. The treatment window member can be disposed between a field lens of the scanning resonance mirror mechanism and the treatment surface.

In some embodiments, the laser is a continuous semiconductor laser.

In some embodiments, the treatment handle further includes a scanning resonance mirror mechanism for causing the laser to have a scanning output, via the optical path and scanning the laser light in accordance with a program of pulse widths and light spot positions set by the control system.

In some embodiments, the control system is configured to control the scanning resonance mirror mechanism to perform a pulse width modulation of in a range of 0.05 ms-1 s.

In some embodiments, the laser output has a wavelength of 500 nm to 1200 nm.

In some embodiments, the range of the spot area adjusted by the scanning resonance mirror mechanism is 0.001 mm² to 1000 cm².

In some embodiments, the laser light spot output by the scanning resonance mirror mechanism is a circular spot or a linear spot.

In some embodiments, the treatment window member is configured to output laser light in a collective pattern.

In some embodiments, the treatment window member is detachably connected to the treatment handle.

In some embodiments, the treatment window member has an aperture control.

In some embodiments, the treatment handle has a detachable connection with the optical path and the electrical circuit on the treatment main portion.

In some embodiments, the detachable connection comprises a rotary disassembly-assembly connection, and a slotted disassembly-assembly connection.

Embodiments disclosed herein can have one or more of the following advantages: optical-fiber-coupled semiconductor laser can be adopted as the light source, greatly reducing the equipment cost while improving the stability of the equipment. The apparatus not only can treat the pigment diseases, but also can treat the vascular diseases such as chloasma, facial flushing, blood vessel dilation, etc. In addition, using the scanning resonance mirror mechanism, pulse width and spot area can be modulated for the treatment of different pigmented diseases.

In another aspect, an optical scanning treatment apparatus is provided, including: a treatment scaffold having at least a vertical treatment frame; a treatment head mounted on the vertical treatment frame and disposed perpendicularly to the vertical treatment frame; a drive motor disposed over the treatment scaffold and connected to the treatment head; a controller connected at least to the drive motor, and configured to move laterally or longitudinally the treatment head at the vertical treatment frame.

In some embodiments, in the optical scanning treatment apparatus, a control panel is further provided and connected to the controller, to set the movement range and the movement speed of the treatment head.

In some embodiments, in the optical scanning treatment apparatus described above, the treatment head includes at least a light source, a light focusing device and a shaping mechanism, the light source being provided at a first front end of the light-focusing surface. The shaping mechanism is disposed at a second front end of the light-focusing surface, and the first front end is between the second front end and the light-focusing surface.

In some embodiments, in the optical scanning treatment apparatus described above, the light focusing device is an ellipsoidal reflecting surface.

In some embodiments, in the optical scanning treatment apparatus described above, the first front end is located at the focal point of the ellipsoidal reflecting surface.

In some embodiments, in the optical scanning treatment apparatus described above, the light source is a UVB ultraviolet light source.

In some embodiments, in the optical scanning treatment apparatus described above, the UVB ultraviolet light generated by the light source includes any one of a 308-nm excimer light source, an LED array light source, a VCSEL array light source, a UVB metal halogen lamp, or a UVB fluorescent lamp.

In some embodiments, in the optical scanning treatment apparatus described above, the shaping mechanism is a lenticular lens.

In some embodiments, in the optical scanning treatment apparatus described above, the lateral coverage width of the light source is 40 cm to 60 cm.

In view of the above, various embodiments disclosed herein can have the following advantages: a large area of rapid treatment (e.g., a side of the whole body) can be realized. Targeted treatment (e.g., treatment area selection control), and output dose optimization options for different body parts can also be realized. Advantages of the 308-nm excimer light targeting and rapid treatment and the 311-nm systemic large area treatment can be combined. In addition, output doses for various body parts can be optimized.

### BRIEF DESCRIPTION OF DRAWINGS

To more clearly illustrate the embodiments of the disclosure, the following is a brief description of the drawings, which are for illustrative purpose only. For those of ordinary skills in the art, other drawings of other embodiments can become apparent based on these drawings.

FIG. 1 is a schematic diagram of an apparatus for achieving pigment and vascular therapy using a low power laser according to some embodiments;

FIG. 2 is a schematic diagram of an optical scanning treatment system according to some embodiments;

FIG. 3 is a schematic view of a treatment head in the optical scanning treatment system according to some embodiments; and

FIG. 4 is a schematic diagram of an optical scanning treatment apparatus in some other embodiments.

### DETAILED DESCRIPTION

In the following, with reference to the drawings of various embodiments disclosed herein, the technical solutions of the embodiments of the disclosure will be described in a clear and fully understandable way. It is obvious that the described embodiments are merely a portion but not all of the embodiments of the disclosure. Based on the described embodiments of the disclosure, those ordinarily skilled in the art can obtain other embodiment(s), which come(s) within the scope sought for protection by the disclosure.

It should be noted that structures, proportions, sizes, etc. shown in the drawings shown in the drawings are intended to cover the same information as those set forth in the specification and are not to be construed, as being understood to those skilled in the art, to be limiting the invention. Modification of any structure, change of the proportionality or adjustment of the size, shall remain within the scope of the present invention without affecting the efficacy and achievable effect of various embodiments disclosed herein. The terms quoted in the present specification, such as “up,” “down,” “left,” “right,” “middle,” and “one,” are merely for illustrative purposes. The change or adjustment of its relative relationship, when not substantially changing the technical content, is also considered as within the scope of the invention.

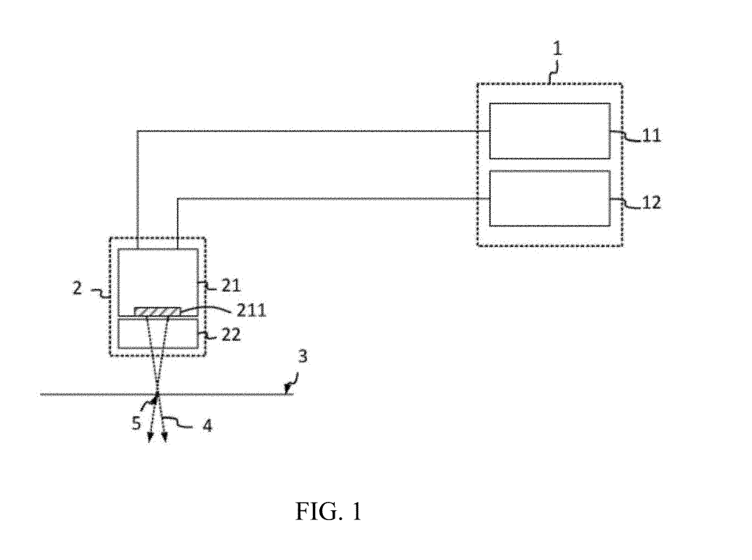

Referring to FIG. 1, embodiments disclosed herein provide an apparatus for achieving pigment and vascular therapy using a low-peak-power laser. The apparatus can include, as shown, a treatment main portion 1 and a treatment handle 2, wherein the treatment main portion 1 comprises a laser 11 for outputting laser light, and a control device such as a controller 12 for performing laser treatment control. In some embodiments, the laser 11 can be a semiconductor laser.

The treatment handle 2 can be connected to the treatment main portion 1 through an optical path and an electrical circuit. The treatment handle 2 includes a scanning resonance optical structure 21, configured to receive, via the optical path, the laser beam 4. The scanning resonance optical structure 21 is also configured to control the scanning of the output of the laser beam 4 in accordance with a program of pulse widths and positions of the light spot 5 set in the control device 12.

The treatment handle 2 can further include a treatment window member 22 configured to adjust the surface area of the light spot 5 of the laser beam 4 at the treatment surface 3. The treatment window member 22 can be disposed between the field lens 211 of scanning resonance optical structure 21 and the treatment surface 3.

Because the pigment treatment requires both the energy density and the pulse frequency of the laser, and the higher requirement of the energy density means that the laser 11 is required to have a higher power, the power increase will bring about a large cost increase. Therefore, the apparatus can use a conventional laser with a relatively low power, such as a semiconductor laser, which is inexpensive.

By confining the surface area of the light spot 5 of the light emitted by the laser in order to increase the energy density in the smaller unit area, and then using the scanning resonance mirror structure 21 to scan the laser output, to thereby realize a large area of pigment targeted treatment. Compared with existing technologies, the approach described above according to some embodiments disclosed herein can have lower cost, lower failure rate, be more practical.

In some embodiments, the scanning resonance optical structure 21 can function as a pulse width modulator to perform a pulse modulation width of 0.05 ms-1 s, and the specific pulse width may be determined based on the power treatment requirement of the laser.

In some embodiments, the laser can be a continuous semiconductor laser for a continuous output of the laser beam. For example, the laser output of the laser can have a wavelength of from 500 nm to 1200 nm. Laser light at a wavelength within this range is advantageous for pigment treatment, such as chloasma, which is particularly sensitive to this range of laser light, having selective absorption, so as to achieve the goal of treatment.

In some other embodiments, a pulsed laser can be adopted. The pulse widths of the laser output can be modulated.

In some embodiments, different applications may need different wavelengths of laser, and the specific device of some embodiments employs a continuous output laser, in the case of pigment-related diseases, with a higher band of melanin absorption, specifically 500 nm to 1100 nm, preferably between 700 nm and 1064 nm. In the treatment of wrinkle-related skin aging symptoms, a band having a higher water absorption value is used, specifically 900 nm to 10600 nm, more preferably 1400 nm to 3000 nm. In the treatment of vascular diseases, a higher band of hemoglobin absorption is used, specifically between 500 nm and 1100 nm, more preferably between 500 nm and 650 nm.

In some embodiments, in addition to the use of a less expensive semiconductor laser to provide a laser light source, it is also advantageous to adjust the spot area of the output laser using the treatment window member 22. For example, if a higher energy density is needed, the area of the spot can be adjusted so that the energy density in the area of the spot is high enough to meet the requirements. The laser light spot can be turned onto the target of the treatment area for scanning in accordance with the size of the spot area to achieve the therapeutic requirement.

If a lower energy density is desired, the spot area can be adjusted to be larger. Similarly, the scanning of the laser beam can be adjusted accordingly based on the laser spot area. In some embodiments, the adjustment of the spot size is achieved by adjusting the distance between the bottom of the treatment window member 22 and the field lens 211. As such, the treatment window member can have an adjustable focal length.

In some embodiments, because the range of the adjusted spot area using the treatment window member 22 is limited, the treatment window member 22 can be provided in a removable form in order to expand the range of the treatment. For example, the treatment window member 22 can be provided as individual components detachably connected to the treatment handle 2 or the scanning resonance optical structure 21 to cooperate with the scanning resonance mirror structure 21 to output laser light. Specifically, the detachable connection can be fixed for rotation, or may also be a slot fixing method or the like.

For example, the spot area of a treatment window member 22 is adjusted in the range of 0.01 mm² to 700 cm², and if the spot area needs to be 0.001 mm², the treatment window member 22 can be replaced to achieve such a smaller spot size. In some embodiments, the treatment window member 22 is capable of adjusting the area of the spot in the range of 0.001 mm² to 1000 cm². Of course, the selection of the treatment window member 22 is also related to the power of the laser, as long as the energy density of the selected laser on the spot of the adjustment range can meet the therapeutic requirements. As such, this arrangement can also provide more choices for the laser.

In some other embodiments, the treatment window member 22 has an aperture control for occluding or shaping the spot of the non-therapeutic area, and avoiding the risk that the software performs a pattern scan out of control. Especially in the treatment near the eye areas, out of control pattern output may lead to eye blindness.

In some embodiments, the light spot of the scanning resonance optical structure 21 can be a circular spot, or a linear spot. It is noted that although the phrase “scanning resonance optical structure” is used, the structure 21 can employ one or more mirrors, one or more lenses to achieve the optical effects as desired, not necessarily with “resonance” of light to realize the control of the light spot. As such, the structure 21 can also be referred to a scanning optical structure 21 in general.

In some embodiments, the scanning optical structure 21 outputs laser light in a geometric pattern, and the pattern is controlled by combining the adjusted light spot and the scanning.

It should be noted that the controller according to some embodiments can be a control system, a control module, or a control circuit board. The implementation of the controller may be software based, hardware based, or a combination of hardware and software. In some embodiments, the control system 12 can control the scanning optical structure 21 to adjust the light spot 5 as a circular light spot, a dot light spot, or a linear light spot. For example, to achieve the linear light spot output from the laser, the scanning optical structure 21 can have its X direction or Y direction laser output adjusted. In some embodiments, the laser output can have a geometrical shape or pattern. Adjusting the light spot to realize a combinational pattern output can be realized with the control system 12 through a software program.

In view of the above, various embodiments disclosed herein adopt an optical fiber coupled semiconductor laser as the light source, can greatly reduce the equipment cost while improving the stability of the equipment. The apparatus not only can treat pigmented diseases, but also can treat vascular diseases such as chloasma, facial flushing, vascular dilatation, etc. The scanning optical structure 21 can have pulse width and spot area adjustment, thereby carrying out the treatment of different pigment diseases. Therefore, various embodiments disclosed herein can effectively overcomes the shortcomings of the prior art and have a high degree of flexibility.

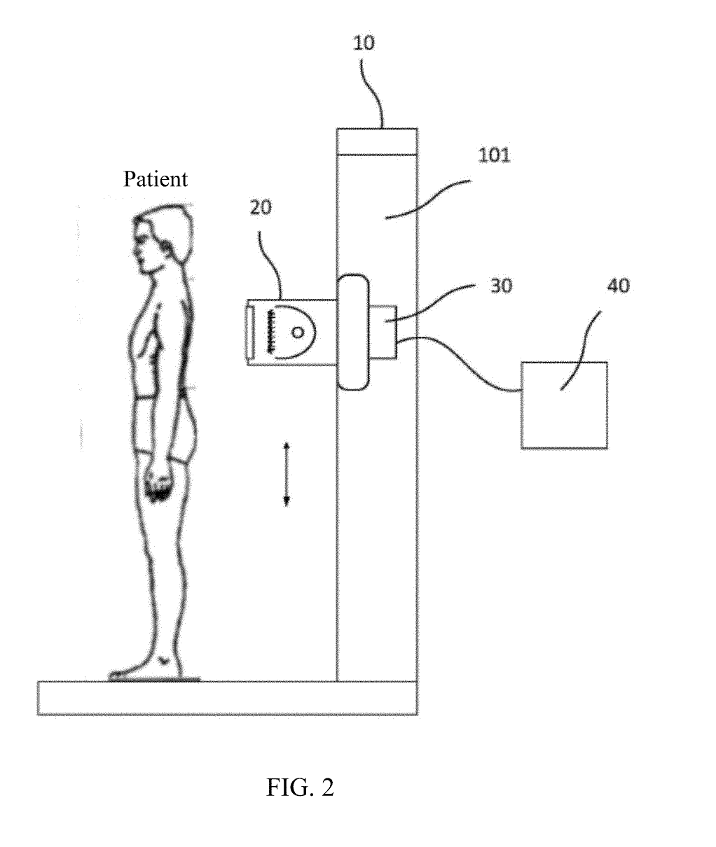

The apparatus described above can be combined with, or part of, various other treatment systems. Referring to FIG. 2, for example, a schematic view of an optical scanning treatment system is provided according to some embodiments. The system includes: a treatment scaffold 10 having at least a vertical treatment frame 101; a treatment head 20 mounted on the vertical treatment frame 101, and a drive motor 30 disposed on the treatment scaffold 10 and connected to the treatment head 20; a controller 40 connected at least to the drive motor 30 for driving the treatment head 20 to be move horizontally and/or longitudinally at the vertical treatment frame 101.

In some embodiments, the treatment head 20 can be, or include some or all components of, the treatment apparatus illustrated in FIG. 1.

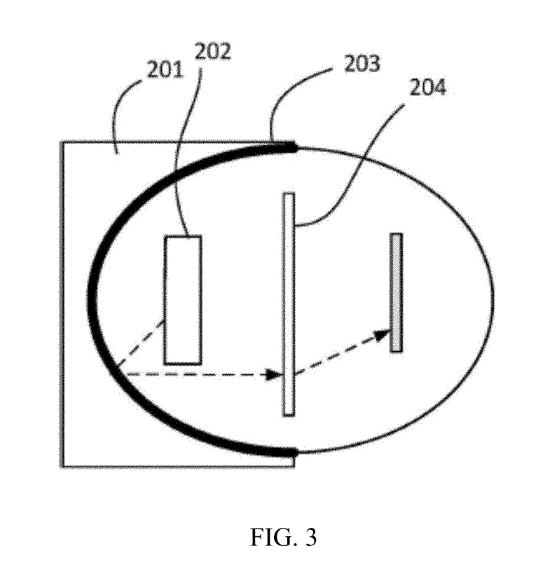

In some other embodiments, as seen in FIG. 3, the treatment head 20 includes a housing 201, a light source 202, a light collection or focusing device 203, and a light shaping mechanism 204. The light source 202 can be disposed at a first front end of a light focal surface of the light focusing device 203. The light shaping mechanism 204 can be disposed at a second front end of the light focal surface of the light-focusing device 203. The first front end is between the second front end and the light focal surface.

In the treatment head 20, the light focusing device 203 can be an ellipsoidal or convex reflector, wherein the first front end is located at the focal point of the ellipsoidal or convex reflector so that the optical performance can be optimal according to some embodiments.

In some embodiments, in the optical scanning treatment system, the light source can be a UVB ultraviolet light source. Ultraviolet light can be divided into PUVA, UVB, UVAl, and so on. In sonic embodiments, UVB ultraviolet light is used as a light source. The UVB can be divided into two modes, high energy UVB, and low energy UVB, specifically divided into the 308-nm excimer light/laser (high energy), and the 311-nm light (low energy, such as low energy fluorescent light). High-energy UVB has the advantage of good efficacy, but with the shortcomings of the treatment area being small, costing more time of the doctors and patients.

Further, because the diverging angle of the light source can be too large, it may be needed to perform the shaping and focusing of the output beam. In some embodiments, the shaping mechanism can include a cylindrical lens disposed in front of the treatment surface, focusing the light as a line or a smaller area. In some embodiments, the shaping mechanism can employ a cylindrical convex lens.

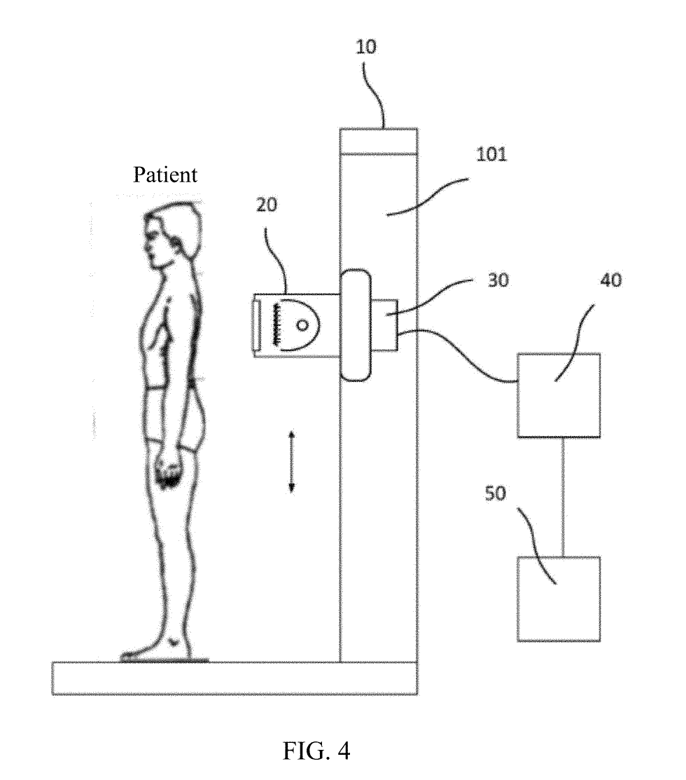

In some embodiments, as shown in FIG. 4, an optical scanning treatment system can include: a treatment scaffold 10 having at least one vertical treatment frame 101; a treatment head 20, a drive motor 30 disposed on the treatment scaffold 10 and connected to the treatment head 20; a controller 40 connected to the drive motor 30, and a control panel 50 for connecting the controller 40 to set the movement range and the movement speed of the treatment head 20 based on a predetermined treatment program. The control panel 50 can set the movement range and the movement speed of the treatment head 20 with respect to the vertical treatment frame 101.

Since the treatment head is moved in such a way as to move on the treatment scaffold and the speed is controllable, it is possible to set the treatment range (e.g., the range of the scanning) of the treatment head 20 by the control panel 50, and may be set to scan from top to bottom, or from bottom to top.

In some embodiments, the treatment dose may also be set. For example, the treatment of different body parts may need different doses. For example, the shoulder treatment may need a dose of 3 J/cm², the back may need a treatment dose of 2.5 J/cm², the specific performance of the moving speed in different parts may vary as well. For example, the movement speed for treating the back portion is 3 mm/s, and the moving speed for treating the shoulder is 2.5 mm/s. In addition, the scan mode can be set to be a slow scan, or a fast scan. The transverse coverage width of the light source can be 40 cm to 60 cm, so that the setting may be substantially the same as the back width of the human body.

For example, as shown in Table 1 below, each body part can have different treatment dose ratios, and the amount of treatment dose can be determined by the speed of movement of the treatment head. Different body parts require different doses of treatment. In the course of treatment, the process can be divided into the front and the back, by controlling the treatment head scanning speed to achieve different dose output. The specific scanning speed difference is the reciprocal of the corresponding ratio, that is, the scanning speed of the shoulder is X/0.8, the back is X/0.7, the waist is X/0.97 . . . , where X is the adjustment coefficient, to be adjusted based on the required dose.

TABLE 1

| Position | Ratio | Position | Ratio |
|---|---|---|---|
| Shoulder | 0.8 | Shoulder | 0.8 |
| Back | 0.7 | Back | 0.8 |
| Waist | 0.97 | Waist | 1.0 |
| Thigh posterior | 1.85 | Thigh anterior | 1.85 |
| Calf posterior | 2.3 | Calf anterior | 2.3 |

In some embodiments, the advantages of the parallel scan mode movement can include that: first, it can avoid blisters caused by the over-treatment of the same target for a continuous of time; second, the scanning speed can be programmed, that is, the speed of movement of the light source, the speed of the body center portion can be moved based on desired speeds. In addition, the scanning range (e.g., height) can be controlled, and according to the regional selection, scanning speed of 0.1 mm/s-100 mm/s can be selected. The variations can be defined, for example, the scanning speed can be in the range of 3 times, accurate to 0.1. In a specific implementation, the scanning speed can vary by a factor of 2.5 times, etc.

In some embodiments, the scanning of the laser beam or laser spot is a combination of the light beam scanning described above with respect to FIG. 1, and the mechanical scanning provided by the drive motor and the drive controller described above with respect to FIG. 2.

In view of the above, various embodiments provide a design of a 308-nm treatment system for large light spots while reducing the difficulty of the operation of the physician and the time of treatment of the patient. Therefore, various embodiments can effectively overcome the shortcomings of the prior art and have a high degree of flexibility.

All references cited herein are incorporated by reference in their entirety. Although specific embodiments have been described above in detail, the description is merely for purposes of illustration. It should be appreciated, therefore, that many aspects described above are not intended as required or essential elements unless explicitly stated otherwise. Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. An apparatus for pigment and vascular treatment, comprising:
a treatment main portion including a low-peak-power laser configured to output a laser light spot;
a controller configured to perform laser treatment control; and
a treatment handle connected to the treatment main portion via an optical path and an electrical circuit;
the treatment handle including:
a scanning resonance optical structure optically coupled to the laser via the optical path and configured to cause the laser to have a scanning output in accordance with a program of pulse widths and light spot positions set by the controller; and
a treatment window member coupled to an output end of the scanning resonance optical structure and configured to adjust a surface area of the laser light spot on a treatment surface;
wherein the treatment window member is disposed between a field lens of the scanning resonance optical structure and the treatment surface.

2. The apparatus of claim 1, wherein the laser is a continuous semiconductor laser.

3. The apparatus of claim 2, wherein the scanning resonance optical structure is configured for position modulation of the laser light spot and pulse width modulation as controlled by the controller.

4. The apparatus of claim 3, wherein the pulse width modulation has a range of 0.05 ms-1 s.

5. The apparatus of claim 1, wherein the laser has an output wavelength of 500 nm to 1200 nm.

6. The apparatus of claim 1, wherein the laser light spot has an area adjusted by the scanning resonance optical structure in a range of 0.001 mm² to 1000 cm².

7. The apparatus of claim 1, wherein the laser light spot is a circular spot or a linear spot.

8. The apparatus of claim 7, wherein the treatment window member is configured to output the laser light spot in a collective pattern.

9. The apparatus of claim 7, wherein the treatment window member is detachably connected to the treatment handle.

10. The apparatus of claim 9, wherein the treatment window member has an aperture control.

11. The apparatus of claim 9, wherein the treatment handle is detachably coupled with the optical path and the electrical circuit.

12. The apparatus of claim 11, wherein the treatment handle is detachably coupled with the optical path and the electrical circuit via a rotary disassembly-assembly connection or a slotted disassembly-assembly connection.

13. A light treatment system, comprising:
a treatment scaffold having at least a vertical treatment frame;
a treatment head mounted on the vertical treatment frame and disposed perpendicularly to the vertical treatment frame;
a drive motor disposed over the treatment scaffold and connected to the treatment head;
a drive controller connected at least to the drive motor, and configured to move laterally or longitudinally the treatment head at the vertical treatment frame;
wherein the treatment head includes:
a treatment main portion including a by a low-peak-power laser configured to output a laser light spot;
a controller configured to perform laser treatment control, wherein the laser is a semiconductor laser; and
a treatment handle connected to the treatment main portion via an optical path and an electrical circuit;
the treatment handle including:
a scanning resonance optical structure optically coupled to the laser via the optical path and configured to cause the laser to have a scanning output in accordance with a program of pulse widths and light spot positions set by the controller; and
a treatment window member coupled to an output end of the scanning resonance optical structure and configured to adjust a surface area of the laser light spot on a treatment surface;
wherein the treatment window member is disposed between a field lens of the scanning resonance optical structure and the treatment surface.

14. The system of claim 13, further comprising a control panel coupled to the drive controller and configured to set a movement range and a movement speed of the treatment head.

15. The system of claim 14, wherein the laser comprises a 308-nm excimer laser.

16. The system of claim 14, wherein the laser comprises a semiconductor laser.

17. The system of claim 16, wherein the semiconductor laser is a continuous laser.

18. The system of claim 16, wherein the laser is a pulsed laser.

19. The system of claim 13, wherein the system is configured to treat pigment-related diseases, and wherein the laser has a wavelength range between 700 nm and 1064 nm.

20. The system of claim 13, wherein the system is configured for treatment of vascular diseases, and wherein the laser has a wavelength range between 500 nm and 650 nm.
